(19) United States
(12) Patent Application Publication
(10) Pub. No.: US 2022/0309897 A1
(43) Pub. Date: Sep. 29, 2022

(54) Wearable Safety Device for Swimming

(71) Applicants: Rick Bender, Topeka, KS (US); Julie Bender, Topeka, KS (US)

(72) Inventors: Rick Bender, Topeka, KS (US); Julie Bender, Topeka, KS (US)

(21) Appl. No.: 17/528,538

(22) Filed: Nov. 17, 2021

Related U.S. Application Data
(60) Provisional application No. 63/165,933, filed on Mar. 25, 2021.

(51) Int. Cl. G08B 21/08; G08B 3/10; A61B 5/00; A61B 5/0205

(52) U.S. Cl. CPC G08B 21/088; G08B 3/10; A61B 5/0002; A61B 5/02055; A61B 5/681

(57) ABSTRACT

This present invention relates to a safety wearable device such as a band for wrist, ankle, or neck, which is designed to alert others if an individual is drowning or struggling in a pool or other body of water. The safety wearable device comprises a display screen, GPS chipset, and various other sensors to monitor the vital signs of the wearer, and in case the wearer is submerged for an extended period of time in the body of water, the device is used to alert parents, caretakers or other individuals having a companion software application installed on their electronic devices. The safety wearable device can also be used to locate children or other individuals who wander off or become lost.

### CROSS-REFERENCE TO RELATED APPLICATION

The present application claims priority to, and the benefit of, U.S. Provisional Application No. 63/165,933, which was filed on Mar. 25, 2021 and is incorporated herein by reference in its entirety.

### FIELD OF THE INVENTION

The present invention relates generally to a field of wearable devices. More specifically, the present invention relates to a swim-safe wearable device that is designed to alert others when a user of the wearable band is drowning or struggling in a pool, lake or other body of water. The swim-safe wearable device of the present invention is used to detect dangerous situations, such as the act of drowning of a child in a body of water, or to identify the exact location of an individual with dementia that suddenly goes missing. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, drowning is one of the most common causes of accidental death in young children. Drowning is also a major risk of death in adults and older children. Young children can easily fall into a body of water, such as a swimming pool, or wander into the ocean and end up drowning. Even persons trained in swimming can become victims to drowning in some circumstances. Often young children swim under the supervision of parents, care takers, lifeguards, swimming coaches or other adults, but it is not unusual for such parents, care takers, lifeguards, swimming coaches or other adults to become distracted by other things, such as conversation with others. Once distracted, people may be unable to detect if their children are in distress in the water, which may lead to serious injury or death.

There are different types of devices and systems that have been suggested to avoid these tragedies, but they fail to alert the child's guardians early enough to be effective. Some devices or systems fail to accurately notify a user to a water danger or water emergency, or fail to provide enough advanced warning of the child drowning or struggling in a pool, lake or other body of water. Devices such as emergency alert devices are also known in the prior art that provide to the wearer the ability to press a panic button. These devices have no other function than to relay a call to a service, which can bring help to the person's location if needed. However, the device requires the wearer to press the panic button, and the wearer may not always be able to press the panic button. In a situation when the wearer is drowning in a body of water, the wearer may be unconscious and may not be able to press the panic button him/herself, thereby failing to inform others in case of emergency situations.

Additionally, in case children or other individuals wander off, their parents, guardians or the like may be unable to locate and find them. People may be unable to find the exact location of the children or other individuals to keep them safe. Children or elderly people or other individuals suffering from dementia have a tendency to become lost. In such situations, it becomes too difficult to find lost individuals and/or track their path. A safety solution to overcome such situations is required in the state of the art.

Therefore, there exists a long felt need in the art for a safety wearable device that provides real-time alerts or notifications to parents, care takers, lifeguards, swimming coaches or others in case their children or any other person is drowning or struggling in any body of water. There is also a long felt need in the art for a safety wearable device that does not require the wearer to manually press any button to inform their supervisors of any pending tragedy or injury. Further, there is a long felt need in the art for a safety wearable device that sends location information of the wearer, in case the wearer is drowning in a swimming pool, lake or other body of water. Furthermore, there is a long felt need in the art for a safety wearable device that enables individuals to track their children or other individuals who have lost their way, in order to keep them safe. Additionally, there is a long felt need in the art for a safety wearable device that enables individuals to locate people who are suffering from dementia and may become lost. Finally, there is a long felt need for a safety wearable device that affords parents and caretakers peace of mind that at-risk individuals can be quickly located.

The subject matter disclosed and claimed herein, in one embodiment thereof, comprises a safety wearable device in the form of a flexible wrist band or alternatively in the form of a bracelet, an ankle band or a necklace that is designed to provide real-time audio alerts or notifications, or visual alerts when a user of the wearable band is drowning or struggling in a pool, lake or other body of water. The safety wearable device is made of an elastic and flexible material such as silicone or rubber. More specifically, the safety wearable device is preferably comprised of one or more sensors that may detect the submerged timing, location and one or more vital signs of the wearer of the band when the wearer enters the swimming pool, lake or other body of water. The band preferably comprises at least one or more holes, a loop or an annular structure, or a fastener that engages in one of the holes to secure the band. Additionally, the band of the present invention can be used to locate individuals suffering from dementia who have a tendency to become lost.

In this manner, the novel safety wearable device of the present invention accomplishes all of the forgoing objectives, and provides a relatively safe, easy, convenient and effective solution to detect a drowning individual, and provide an alert or push notification to persons in the surrounding area, and to allow the notified persons to rescue the drowning individual in time. Additionally, the safety wearable device can be used to track the location of children or individuals who wander off, or are suffering from dementia.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a wearable band device for the detection of a wearer of the band device in a drowning or near-drowning situation and ‘pushes’ a notification to one or more connected electronic devices. The wearable device comprises: a location sensor for detecting location of the wearer; a wireless transceiver for wirelessly connecting to one or more said electronic devices; an accelerometer for detecting orientation and depth of the wearer in water; a water-detecting sensor for detecting water and triggering a timer circuit to start counting time; the timer circuit counts the number of seconds for which the wearable band device is submerged in the water, and, a processor to control operations of electronic components of the band device and transmit one or more alerts to the connected electronic devices when the timer circuit counts for a time value longer than a predeterminable timer value. A sound sensor emits a loud audio alert when the timer circuit counts for a time value longer than a predeterminable timer value. The preferred value of the predeterminable timer value can be in the range of ten seconds.

In a further embodiment of the present invention, a system for the detection of a wearer of a wearable safety band in a drowning or near-drowning situation is disclosed and comprises: a wearable safety band designed to be worn on a wrist, neck or ankle by the wearer; a computer-implemented application designed to be installed in one or more electronic devices; the band comprises a location sensor for detecting the location of the wearer; a wireless transceiver for wirelessly connecting to one or more electronic devices; an accelerometer for detecting orientation and depth of the wearer in water; a water-detecting sensor for detecting water and triggering a timer circuit to start counting time; the timer circuit counts the number of seconds for which the wearable band device is submerged in the water; and, a processor to control operations of electronic components of the band device and transmit one or more alerts to the electronic devices when the timer circuit counts for a time value longer than a predeterminable timer value. The one or more electronic devices include computer-implemented applications holding computer-executable instructions allowing a processor of the electronic device to display received alerts wherein the alerts are at least one of an audio alert, a text alert or a visual alert.

In yet a further embodiment of the present invention, a method of rescuing a person from drowning in a body of water is disclosed and comprises: wearing a safety band around a wrist, neck or ankle; pairing the wearable safety band with one or more electronic devices, wherein each of the one or more electronic devices has a software application installed; triggering a timer circuit of the wearable safety band when the person wearing the wearable safety band enters into the body of water; transmitting one or more vital signs of the person to the one or more paired electronic devices in real time; and, transmitting an alert to the one or more paired electronic devices when the timer circuit counts for a time value longer than a predeterminable timer value indicating that the person is in a near drowning condition allowing nearby people to rescue the person.

In still yet another embodiment of the present invention, a method is disclosed for providing an alert between at least two mobile users and enabling an emergency rescue. The method comprises: pairing one mobile wearable electronic device to at least one other mobile electronic device; wearing the mobile wearable electronic device on a person of one mobile user, wherein the mobile wearable electronic device is a band worn in direct contact with the person of the one mobile user; monitoring a plurality of inputs of the one mobile user, wherein the plurality of inputs comprises information selected from a group consisting of a submersion, a submersion time duration, a vital sign, a submersion depth, a location and a temperature, and wherein the monitoring of the plurality of inputs is automatically actuated when the mobile wearable electronic device is submerged in water; comparing at least one of the inputs to a predeterminable limit; determining when at least one of the inputs is beyond the predeterminable limit; and transmitting an alert to the at least one other mobile electronic device when at least one of the inputs is beyond the predeterminable limit, wherein the transmitting the alert is an automatic push alert to the at least one other mobile electronic device.

It should be noted that the wearable band of the present invention can connect to a plurality of electronic devices and an alert can be simultaneously transmitted to the plurality of electronic devices. The software application allows the user to display the alert or notification on a display of the electronic device.

The device and method of the present invention activates an alarm when the wearable device is submerged for a pre-defined number of seconds, for example ten seconds, along with measuring vital signs and/or environmental variables while the wearable device is submerged, activated, and worn by a person. The timer value and the vital signs are processed in real time for the detection of a drowning or a near-drowning situation of the person.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed, and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the swim-safe wearable band of the present invention which is designed to alert others if a wearer is drowning or struggling in a pool or other body of water in accordance with the disclosed structure;

FIG. 2 illustrates a schematic diagram of connection of one potential embodiment of the swim-safe wearable band of the present invention with a companion smartphone application installed in an electronic device in accordance with the disclosed structure;

FIG. 3 illustrates a block diagram showing exemplary components and sensors of one potential embodiment of the swim-safe wearable band of the present invention in accordance with the disclosed structure;

FIG. 4 illustrates a flow diagram showing an operation of one potential embodiment of the swim-safe wearable band of the present invention for generating an alert in accordance with the disclosed structure;

FIG. 5 illustrates a perspective view of a child wearing one potential embodiment of the swim-safe wearable band of the present invention submerged in the water in accordance with the disclosed structure;

FIG. 6 illustrates a flow diagram showing steps of the receiving alert and other vital information of a wearer by an electronic device from one potential embodiment of the swim-safe wearable band of the present invention in accordance with the disclosed structure;

FIG. 7 illustrates a perspective view showing an alert received on the smartphone application installed in an electronic device from one potential embodiment of the swim-safe wearable band of the present invention in accordance with the disclosed structure; and

FIG. 8 illustrates a perspective view showing one potential embodiment of the swim-safe wearable bands of the present invention in various color option in accordance with the disclosed structure.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

The process of drowning may be detected by monitoring the activities of individuals, especially young children, and many more who are in a body of water. There are several indicators or signs that may be used to determine whether a drowning may be taking place. One purpose of the device disclosed herein is detecting these potential signs of drowning, and providing an audio alert, text alert, or other warnings to nearby mobile phones in order to initiate a rescue.

As noted above, there exists a long felt need in the art for a safety wearable device that provides real-time alerts or notifications to the parents, care takers, life guards, swimming coaches or others in case their children or any other person is drowning or struggling in any body of water. There is also a long felt need in the art for a safety wearable device that does not require the wearer to manually press any button to inform their supervisors of any tragedy or injury. Further, there is a long felt need in the art for a safety wearable device that sends the location information of the wearer in case the wearer is drowning in a swimming pool, lake or other body of water. Furthermore, there is a long felt need in the art for a safety wearable device that enables individuals to track their children or other individuals who have lost their way and allows them to keep them safe. Additionally, there is a long felt need in the art for a safety wearable device that enables individuals to locate people who are suffering from dementia and are lost. Finally, there is a long felt need for a safety wearable device that affords parents and caretakers peace of mind that at-risk individuals can be quickly located.

The present invention, in one exemplary embodiment, is a novel wearable band device for the detection of a wearer of the band device in a drowning or near-drowning situation and “pushes” a notification to one or more connected electronic devices. The device comprises: a location sensor for detecting the location of the wearer; a wireless transceiver for wirelessly connecting to one or more of the electronic devices; an accelerometer for detecting orientation and depth of the wearer in water; a water-detecting sensor for detecting water and triggering a timer circuit to start counting time; the timer circuit counts the number of seconds for which the wearable band device is submerged in the water; and, a processor to control operations of electronic components of the band device and transmits one or more alerts to the connected electronic devices when the timer circuit counts for a time value longer than a predeterminable timer value. A sound sensor emits a loud audio alert when the timer circuit counts for a time value longer than a predeterminable timer value. The preferred value of the predeterminable timer value can be in the range of ten seconds.

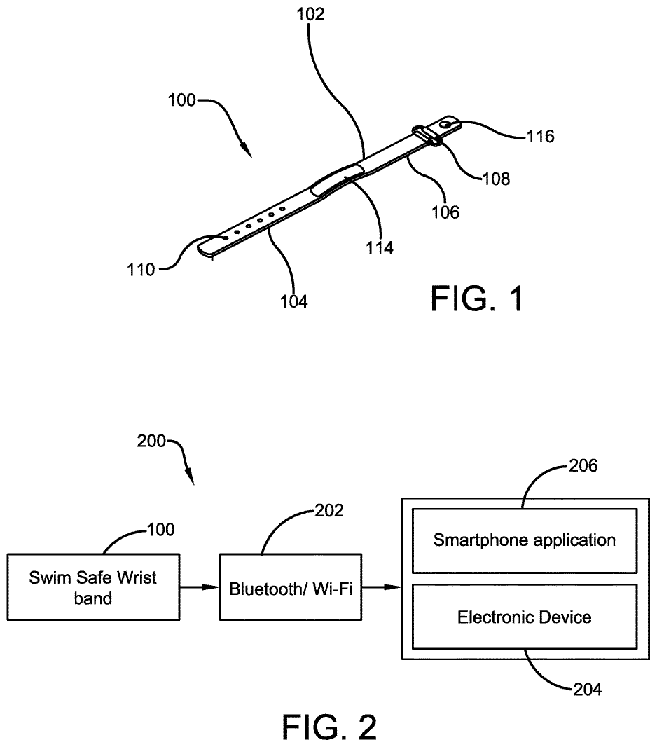

Referring initially to the drawings, FIG. 1 illustrates a perspective view of the swim-safe wearable band of the present invention designed to alert others if a wearer is drowning or struggling in a pool or other body of water in accordance with the disclosed structure. The swim-safe wearable band 100 of the present invention is a wearable device in the form of a flexible wrist band 100, or alternatively in the form of a bracelet, an ankle band or a necklace, which is designed to alert others when a wearer of the safety wearable band 100 is drowning or struggling in a pool, lake or other body of water. The band 100 has a main body 102 and a fastening mechanism 116 such as a wrist buckle, a snap button, a snap closure or a magnetic fastener for securing the band 100 around a body part such as a wrist of the wearer. The main body 102 comprises a digital display 114 configured to display the current time, vital sign information such as oxygen saturation (SpO2) and other similar details. The digital display 114 is preferably positioned in the middle portion of the main body 102 of the band. The position of the display 114 is not limited and can be positioned on other desired locations of the main body 102. The band 100 can be made of an elastic and flexible material such as silicone or rubber. Specifically, the band 100 can be made of an elastically stretchable material such as a Thermoplastic Elastomer (TPE), a Thermoplastic polyurethane Elastomer (TPU), latex or silicone.

The main body 102 extends from both ends of the display 114 to form a first band part 104 and a second band part 106. The first band part 104 is provided with at least one or more holes 110 to receive the fastener 116. The second band part 106 of the band 100 is provided with a loop or an annular structure 108 through which the first wrist band part 104 is allowed to pass for securing the band 100 around a body part such as wrist of the user. As stated, the fastener 116 engages in one of the holes 110 to secure the band 100. The fastener such as a snap closure 116 embeds in one of the holes 110 of the first band part 104 to secure the first band part 104 to the second band part 106 when the end of the first band part 104 passes through the loop, or an annular structure 108.

The digital display screen 114 may have an integrated, waterproof and durable solar panel for charging of the digital screen 114. The solar panel has a plurality of solar cells to absorb solar energy and to convert it into electrical energy. At the rear surface of the digital screen 114, a PCB (Printed Circuit Board) to house electronic components and sensors, and a battery to power the electronic components is present. To customize the band or to change the main body 102, the digital screen 114 of the band 100 may also be replaceable.

It should be noted that all the electronic components of the band 100 are connected through one or more wired circuits within the band 100. The wired circuits are insulated and protected from the moisture and water of a swimming pool, lake, or other body of water. The details of the sensors and other electronic components will be described hereinafter with reference to the figures.

FIG. 2 illustrates a schematic diagram of connection of the swim-safe wearable band of the present invention with a companion smartphone application installed in an electronic device, as per the disclosed architecture. As shown, an alert-generating system 200, using the swim safe wearable band 100, may connect to one or more nearby smartphone devices 204 using a wireless communication medium 202 such as Bluetooth, Wi-Fi or any other similar wireless technology. The smartphone device 204 has a companion software application 206 that receives alerts and notifications from the swim-safe wearable band 100. One or more sensors of the band 100 detect the submerged timing (i.e. duration), location and one or more vital signs of the wearer of the band 100 when the wearer enters a swimming pool, lake or other body of water. The band 100 automatically sends real-time alerts to one or more nearby mobile phones 204, each having installed the companion software application 206. The alert may be activated when the wearable device 100 is submerged for a pre-defined number of seconds such as, for example, ten seconds. A timer value/duration and vital signs may be processed in real time for the detection of a drowning or a near-drowning situation of the wearer. The alert may have information such as a wearer's location, the respiratory and other vital signs detected by the built-in sensors of the band 100 and other similar useful information.

It should be appreciated that parents, care takers, lifeguards and swimming coaches can possess the companion application 206 downloaded on their respective electronic devices 204 to receive real-time alerts of a wearer of the band 100. The location sensor of the band 100 is useful to find individuals that have gotten lost, such as small children, elderly people or those with dementia. The alert or notification shown on the software application 206 may be in the form of a ‘push’ notification, an audio alert or a visual alert.

The electronic device 204 may be any of the terminal devices such as a smart phone, a tablet computer, a laptop, a smartwatch or the like, and a user of the electronic device 204 may be a parent, a teacher, a lifeguard and the like.

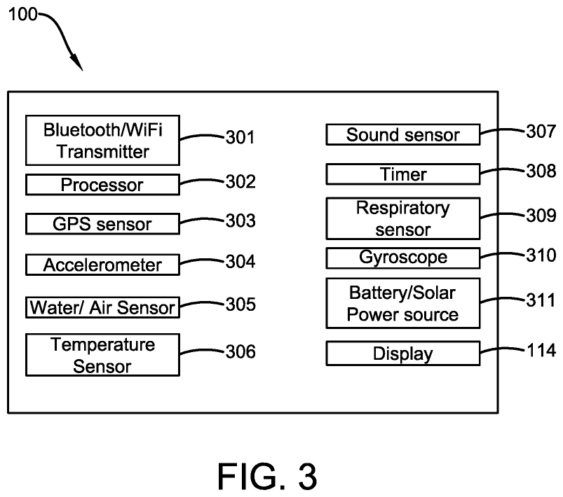

FIG. 3 illustrates a block diagram showing exemplary components and sensors of the swim-safe wearable band of the present invention in accordance with the disclosed structure. As shown, the wearable safety device or swim-safe wearable band 100 comprises a Bluetooth/Wi-Fi transceiver 301 which is a wireless transceiver allowing the swim-safe wearable band 100 to connect to one or more nearby smartphone devices. The Bluetooth/Wi-Fi transceiver 301 allows the swim-safe wearable band 100 to transmit alerts and notifications to a paired electronic device, wherein the alerts and notifications are displayed on the interface of the companion software application. The swim safe wearable band 100 includes a processor 302 to process the signals obtained from the sensors, and also to digitize the analog signals, if analog sensors are utilized. The processor 302 may also perform some rudimentary processing of the sensor data, if desired, for selecting the alerts and other information to be transmitted from the wearable band 100.

Additionally, the swim-safe wearable band 100 includes a GPS sensor 303 which is used to detect the exact location of the wearer, and helps in identifying the location of the wearer if the wearer is lost. It is to be appreciated that the GPS sensor 303 of the band 100 is particularly useful to find individuals that have gotten lost, such as small children, elderly people or those with dementia. The location information of the wearer can enable the paired user(s) of the electronic device to timely rescue the wearer of the wearable device 100 when the user of the electronic device is not far away from the user of the wearable device, and the user of the electronic device can quickly reach a dangerous location of the user of the wearable device. The band 100 can further include an accelerometer 304 which is used to detect motion of the individual and/or detect the orientation and depth of the individual within the body of water.

The band 100 includes a water and/or air detecting sensor 305, which is used to detect when the band 100 is above water, and/or when the band 100 touches or goes below water. The water and/or air detecting sensor 305 can also detect the depth of the swimmer in the body of water. The band 100 further includes a temperature sensor 306 which is used to detect the temperature of the individual and/or the surrounding environment, and a sound sensor 307 that produces an alarm if the water/air sensor 305 detects the depth is more than a predeterminable depth in water such as ten feet, for example. The depth can be predeterminable by the user by using various settings in the band 100, or may be predeterminable during manufacturing of the band 100.

Additionally, the band 100 comprises an internal timer 308 that triggers or starts when the band 100 is submerged under water. If the timer 308 exceeds a predeterminable time period, such as ten seconds, for example, the band 100 automatically transmits a real-time alert or a notification to one or more nearby paired electronic devices, each having the installed software application. If the timer 308 does not exceed the predeterminable time period, no alerts or notifications will be generated to the one or more nearby paired electronic devices, however, selective vital sign information can be transmitted by the band 100. The time period can be predeterminable by the user by using various settings in the band 100, or may be predeterminable during manufacturing of the band 100.

The band 100 can include a respiratory sensor 309 that can detect the breathing rate of the wearer. The respiratory sensor 309 can also detect the sudden gasping for breath, greatly increased or decreased heart rates, or the cessation of breathing, any of which may indicate potential drowning events, and in the case of such an event, the band 100 can generate an alert or notification, along with a loud sound using the sound sensor 307.

The band 100 comprises a battery or a solar power source 311 which is used to power various components of the band 100. The band 100 has a waterproof digital display 114 configured to display current time, depth in water, vital signs information such as SpO2 and other important information. The sensors disclosed above can be powered by embedded batteries, solar cells, collected RF signals or temperature differentials, or can obtain power from any of a number of additional alternatives. The band 100 can automatically start, initiate or actuate once the band 100 touches or enters the water, so that a wearer is not required to manually start the band 100. By utilizing a plurality of the above sensor types, the determination of the existence or absence of a drowning event represented by the drowning symptoms can be made more accurate.

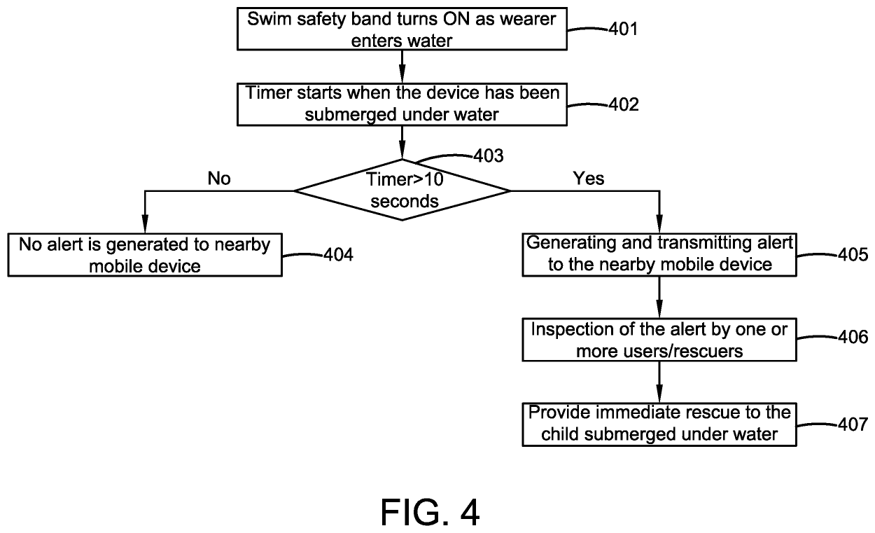

FIG. 4 illustrates a flow diagram showing an operation of the swim-safe wearable band of the present invention for generating an alert in accordance with the disclosed structure. As shown, initially, the swim safety band 100 turns ON (i.e. actuates) as a wearer enters a pool, a lake or other body of water (Block 401). The swim safety band 100 is designed to alert others if the wearer is drowning or struggling in a pool or other body of water. The swim safety band 100 senses and starts the timer when the device is submerged under the water (Block 402). Next, it is determined if the device is submerged in water for an extended period of time, such as ten seconds (Block 403). If the device has been submerged under water for less than a predeterminable timer value, then no alerts or notifications are generated to the one or more nearby mobile phones (Block 404). However, if the band device 100 has been submerged under water for an extended period of time, and the timer value exceeds the predeterminable timer value (Block 403), then a real-time alert or notification is generated and transmitted to the one or more nearby mobile phones, each having installed the paired software application (Block 405). The received alert is seen and reviewed by one or more of parents, care takers, lifeguards or swimming coaches having the paired electronic devices (Block 406). The alert or notification shown on the software application may be in the form of a ‘push’ notification, an audio alert or a visual alert. The alert will show the wearer's exact location and the time they have been in the water. Then, based on the received alerts, the parents, care takers, lifeguards, swimming coaches or other nearby users may provide immediate rescue to the wearer submerged under water (Block 407).

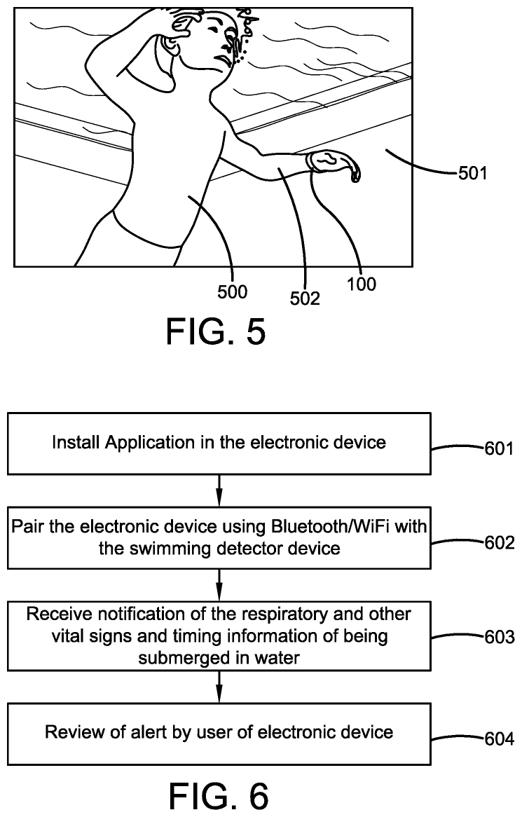

FIG. 5 illustrates a perspective view of a child wearing the swim-safe wearable band of the present invention submerged in water in accordance with the disclosed structure. As shown, a young child 500 is submerged in a body of water 501 such as a swimming pool. The child 500 is wearing the swim-safe wearable band 100 on the wrist 502. The swim-safe wearable band 100 is a preferably a flexible wrist band 100, or alternatively can be a bracelet, an ankle band or a necklace which is designed to alert others when the wearer of the band 100 is drowning or struggling in a pool, lake or other body of water 501. As the child 500 is submerged in the water, the internal timer of the band 100 senses when the band 100 is submerged under water, and in case the band is submerged for an extended period of time (i.e. a period of time that exceeds a predeterminable time period), then one or more real-time alerts or notifications are generated and transmitted to nearby electronic devices with the installed companion software application. The band 100 enables the users to provide immediate help to the child 500 and rescue the child 500 in case the child is drowning in a body of water.

The band 100 prevents a child or other individual from drowning by ensuring others are alerted to the individual's location and predicament, if the user wearing the band 100 is submerged in the water for an extended period of time. The band 100 provides peace of mind to parents and caretakers by allowing them to know the state of the wearer 500 quickly and in real time.

FIG. 6 illustrates a flow diagram showing steps to receive alerts and other vital information of a wearer by an electronic device from the swim-safe wearable band of the present invention in accordance with the disclosed structure. As shown, for receiving real-time alerts or notifications on an electronic device, parents, care takers, lifeguards, swimming coaches, etc., install the companion software application on their respective electronic devices (Block 601). The application can be downloaded from an application store and the application is compatible with the wearable band 100. Next, the electronic device is paired with the swim-safe wearable band 100 using a wireless communication medium such as Bluetooth, Wi-Fi or any other similar wireless technology. For pairing an electronic device with the swim-safe wearable band, the electronic device needs to be in the proximity of the swim safe wearable band (Block 602). The wireless transmitter of the wearable band 100 allows the transmission of alerts and notifications to the paired software application of the electronic device. The electronic device receives notifications including respiratory and other vital signs detected by the one or more sensors, and timing information of the wearer submerged in the water (Block 603). The electronic device companion software application receives the alerts in the form of an alarm, or a visual alert. The alert will show the wearer's exact location and the time they have been in the water. The received alert is seen and reviewed by one or more parents, care takers, lifeguards, swimming coaches, etc., possessing one of the paired electronic devices (Block 604).

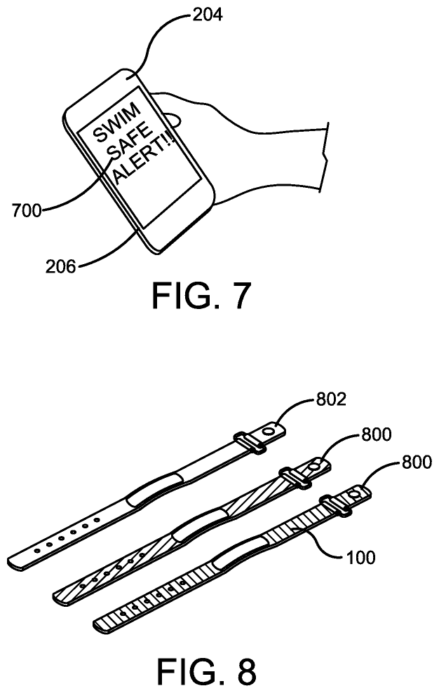

FIG. 7 illustrates a perspective view showing an alert received on the smartphone application installed on an electronic device from the wearable band of the present invention in accordance with the disclosed structure. The paired electronic device 204, such as a smartphone device, has a companion software application 206 installed thereon. The device 204 receives alerts and notifications 700 from the swim-safe wearable band 100 worn by the user while submerged in water. The alert displayed on the paired electronic device 204 shows the submerged timing information i.e., “for ten seconds”, location, and/or one or more vital signs of the wearer of the band when the wearer enters a body of water such as a swimming pool, lake or any other body of water, and is submerged in the body of water for an extended period of time. Parents, care takers, lifeguards, swimming coaches, etc., based on the received alerts, can provide immediate rescue to the wearer submerged under water.

It should be noted that the smartphone application can display additional information that may be useful for providing safety to the wearer. The notification may be displayed on one or more electronic devices.

FIG. 8 illustrates a perspective view showing swim-safe wearable bands of the present invention in various color options in accordance with the disclosed structure. As shown, the swim-safe wearable band 100 of the present invention is available in various color options and sizes 800, 802. The wearable band 100 of various embodiments may be made of an elastic and flexible material such as silicone or rubber, specifically, the band 100 may be made of an elastically stretchable material such as a Thermoplastic Elastomer (TPE), a Thermoplastic polyurethane Elastomer (TPU), latex, or silicone. The specific material of wrist strap body can be any variety of materials outlined above. The band 100 is provided with a loop or an annular structure through which the band is allowed to pass for securing around a body part. In one embodiment, in addition to transmitting alerts to one or more electronic devices, the wearable device 100 may be connected to a central safety system of a campus, such as of a swimming pool, that may provide instructions to lifeguards to protect the wearers in dangerous situation.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “swim-safe wearable band”, “flexible wrist band”, “safety wearable band”, “band”, “wearable safety device”, and “swim safety band” are interchangeable and refer to the swim-safe wearable band 100 of the present invention.

Notwithstanding the forgoing, the swim-safe wearable band 100 of the present invention can be of any suitable size and configuration as is known in the art without affecting the overall concept of the invention, provided that it accomplishes the above-stated objectives. One of ordinary skill in the art will appreciate that the size, configuration and material of the swim-safe wearable band 100 as shown in the FIGS. are for illustrative purposes only, and that many other sizes and shapes of the swim-safe wearable band 100 are well within the scope of the present disclosure. Although the dimensions of the swim-safe wearable band 100 are important design parameters for user convenience, the swim-safe wearable band 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method of providing an alert between at least two mobile users and enabling an emergency rescue, the method comprising the steps of:
pairing one mobile wearable electronic device to at least another mobile electronic device;
wearing said mobile wearable electronic device on a person of one mobile user, wherein said mobile wearable electronic device is a band worn in direct contact with said person of said one mobile user;
monitoring a plurality of inputs of said one mobile user, wherein said plurality of inputs comprises information selected from a group consisting of a submersion, a submersion time duration, a vital sign, a submersion depth, a location, and a temperature, and wherein said monitoring said plurality of inputs is automatically actuated when said mobile wearable electronic device is submerged in water;
comparing at least one of said inputs to a predeterminable limit;
determining when at least one of said inputs is beyond said predeterminable limit; and
transmitting an alert to said at least another mobile electronic device when said at least one of said inputs is beyond said predeterminable limit, wherein said transmitting said alert is an automatic push alert to said at least another mobile electronic device.

2. The method of claim 1, wherein said temperature is selected from a group consisting of a water temperature, an air temperature, and a body temperature.

3. The method of claim 1, wherein said temperature is at least two temperatures selected from a group consisting of a water temperature, an air temperature, and a body temperature.

4. The method of claim 1, wherein said vital sign is selected from a group consisting of a breathing rate, a heart rate, a cessation of breathing, and an SpO2.

5. The method of claim 2, wherein said mobile wearable electronic device is a flexible band adapted to be worn around a wrist of said person of said one mobile user.

6. The method of claim 5, wherein said flexible band comprises material selected from a group consisting of a rubber and a silicone.

7. The method of claim 6, wherein said flexible band is solar powered.

8. The method of claim 1, wherein said alert includes an audible alarm.

9. The method of claim 8, wherein said alert includes a GPS location.

10. The method of claim 9, wherein said at least another mobile electronic device is a mobile phone.

11. The method of claim 1, wherein said mobile wearable electronic device is a flexible band adapted to be worn around a neck of said person of said one mobile user.

12. The method of claim 11, wherein said alert includes an audible alarm.

13. The method of claim 12, wherein said alert includes a GPS location.

14. The method of claim 13, wherein said at least another mobile electronic device is a mobile phone.

15. A method of providing an alert between a plurality of mobile users and enabling an emergency rescue, the method comprising the steps of:
pairing one mobile wearable electronic device to at least two other mobile electronic devices;
wearing said mobile wearable electronic device on a person of one mobile user, wherein said mobile wearable electronic device is a band worn in direct contact around a neck of said person of said one mobile user;
monitoring a plurality of inputs of said one mobile user, wherein said plurality of inputs comprises information selected from a group consisting of a submersion, a submersion time duration, a vital sign, a submersion depth, a location, and a temperature, wherein said monitoring said plurality of inputs is automatically actuated when said mobile wearable electronic device is submerged in water;
comparing at least one of said inputs to a predeterminable limit;
determining when at least one of said inputs is beyond said predeterminable limit; and
transmitting an alert to said at least two other mobile electronic devices when said at least one of said inputs is beyond said predeterminable limit, wherein said transmitting said alert is an automatic push alert to said at least two other mobile electronic devices.

16. The method of claim 15, wherein said temperature is selected from a group consisting of a water temperature, an air temperature, and a body temperature.

17. The method of claim 15, wherein said vital sign is selected from a group consisting of a breathing rate, a heart rate, a cessation of breathing, and an SpO2.

18. The method of claim 15, wherein said alert includes an audible alarm.

19. The method of claim 18, wherein said alert includes a GPS location.

20. A wearable safety device comprising:
a strap;
a display screen;
a GPS chipset; and
a plurality of sensors.
